(19) United States
(12) Patent Application Publication
(10) Pub. No.: US 2022/0008227 A1
(43) Pub. Date: Jan. 13, 2022

(54) METHOD AND DEVICE FOR CONSTRUCTING A PROSTHESIS

(71) Applicant: Ottobock SE & Co. KGaA, Duderstadt (DE)

(72) Inventors: Christian Will, Göttingen (DE); Martin Pusch, Duderstadt (DE); Lars Benjamin Finke, Landolfshausen (DE); Alexander Glier, Göttingen (DE); Juan Pablo MEJIA NINO, Mödling (AT)

(73) Assignee: Ottobock SE & Co. KGAA, Duderstadt (DE)

(21) Appl. No.: 17/294,366

(22) PCT Filed: Nov. 4, 2019
(86) PCT No.: PCT/EP2019/080088

§ 371 Date: May 14, 2021

(30) Foreign Application Priority Data
Nov. 14, 2018 (DE) 10 2018 128 514.0

(51) Int. Cl. A61F 2/76; A61F 2/60

(52) U.S. Cl. CPC A61F 2/76; A61F 2/60; A61F 2002/5092; A61F 2002/607; A61F 2002/608

(57) ABSTRACT

The invention relates to a method for carrying out a prosthesis set-up, wherein multiple components are arranged close to each other, the method comprising the a) providing at least one marking on each of the at least one components, b) arranging various components close to each other, c) detecting a position and/or an orientation of the at least one marking of the closely arranged components by means of at least one optical sensor, d) determining the actual position and/or the actual orientation of the closely arranged components relative to one another using the detected position and/or orientation of the at least one marking, and e) comparing the determined actual position and/or actual orientation with a target position and/or target orientation.

The invention relates to a method for carrying out a prosthesis set-up in which multiple components are arranged close to one another.

Nowadays, many prostheses are made up of different components. For example, a prosthesis for an upper-leg amputee comprises the prosthesis socket, a knee joint, a lower leg element, an ankle joint, and a prosthetic foot. All of these components must be arranged in the right orientation in relation to each other to ensure an optimum force path when the prosthesis is in use and its full functionality. The set-up of the prosthesis constitutes a decisive factor for the comfort and sense of security felt by the wearer of the prosthesis. For example, if the various components are not optimally arranged in relation to each other, i.e. the prosthesis set-up is incorrect, this may lead to insufficient ground clearance in the case of a leg prosthesis, thereby increasing the risk of stumbling. In addition, an incorrect prosthesis set-up may cause an overextension of ligaments and hyperextension, joint pain, back problems or an increased risk of stumbling, for example with an incorrect release time for the swing phase of a step.

EP 0 663 181 A2 therefore discloses an display system in which a wearer of a prosthesis stands on a measuring plate that is fitted with pressure sensors. These are able to determine the center of gravity of the prosthesis wearer's body. A line of body gravity is projected onto the human body of the prosthesis wearer via a laser projection unit that generates a linear beam of light. This is a very effective way to determine the center of gravity line of the body in relation to the joint positions of the prosthesis. In addition, the laser projection line can be moved so that it passes through joint axes of, for example, a knee joint. This renders it possible to determine the movement path and therefore a corresponding measure for any changes to be made in the prosthesis set-up.

However, adjusting the prosthesis set-up based on the determined line of body gravity is not easy for the orthopedic technician.

Therefore, WO 2007/128266 discloses an improved display system with which vertical and horizontal components of the ground reaction force can be detected and the projection device driven on the basis of this data. In this case, both the location and orientation of the ground reaction force are displayed.

However, this system also requires the orthopedic technician setting up the prosthesis to have extensive knowledge of the components to be combined and their technical possibilities. For example, a prosthetic knee joint that enables bending in the stance phase has to be adjusted differently to a prosthetic knee joint without such capabilities in order to ensure optimum treatment of the patient. Consequently, the orthopedic technician must either have extensive specialist knowledge of products from different manufacturers, if applicable, and their ability to be combined, consult appropriate reference works during prosthesis set-up or provide the patient not with the optimal prosthesis, but only with a functioning prosthesis with a correspondingly non-optimal prosthesis set-up.

A device with which the various components of the prosthesis can be aligned in relation to one another is known, for example, from DE 10 2008 024 749 A1. It has the option of aligning different components of a leg prosthesis, such as a knee joint, foot part and/or upper leg socket, in relation to one another and moving in different planes as well as swivelling and tilting about different axes. A corresponding device is marketed by the applicant under the name Pros.A.Assembly.

The invention aims to eliminate these difficulties or at least to reduce them.

The invention solves the problem by means of a method for carrying out a prosthesis set-up, wherein multiple components are arranged close to each other, the method comprising the following steps:
a) providing at least one marking on at least one component,
b) arranging various components close to each other,
c) detecting a position and/or an orientation of the at least one marking of the closely arranged components by means of at least one sensor,
d) determining the actual position and/or the actual orientation of the closely arranged components relative to one another using the detected position and/or orientation of the at least one marking, and
e) comparing the determined actual position and/or actual orientation with a target position and/or target orientation.

While methods known from the prior art require the determination of the ground reaction forces or body's centre of gravity of the respective patient wearing the prosthesis in order to determine whether an already optimal prosthesis set-up or a prosthesis set-up that it yet to be improved has been carried out, the method according to the present invention can also be conducted without the patient. To this end, markings are provided on at least one, but preferably several, of the components to be used, wherein said markings can be detected by the sensor. Some or all of the components to be used are subsequently arranged close to each other, thereby resulting in the prosthesis or a part of the prosthesis. The sensor then detects the markings of these closely arranged components, the position and/or the orientation of the marking being detected by the sensor. Using this data, an electric or electronic control system, in particular an electronic data processing device such as a microprocessor, can determine an actual position and/or an actual orientation of the respective components relative to one another.

For this purpose, the exact position of the at least one marking on the respective components must be known, so that the actual position and/or the actual orientation of the respective components can be deduced from the position and/or orientation of the marking. The actual positions and/or actual orientations of the components determined in this way are compared with corresponding target positions and/or target orientations. As such, deviations can be easily determined without the patient having to wear the prosthesis to do so. The prosthesis set-up phase is therefore easier and shorter for the patient.

Preferably, the prosthesis is set up statically. Alternatively or additionally, the prosthesis can also be set up dynamically.

The prosthesis is preferably set up according to a set-up recommendation. Such a set-up recommendation designates at least one of the components as relevant for adjustment in order to achieve the optimal prosthesis set-up. Preferably, all components that must be adjustable or adjusted in order to achieve the optimal prosthesis set-up are designated as relevant for adjustment. This may well be all components to be used.

In a preferred embodiment of the method, at least one marking is provided on each of the components designated as relevant for adjustment in the set-up recommendation.

The at least one sensor preferably comprises an optical sensor, preferably a camera, preferably for visible light or infrared radiation. The use of a sensor for infrared radiation and, of course, of corresponding markings has the advantage that they are invisible to the human eye, such that any markings on the components are not visually distracting. In principle, however, the selection of the wavelength of the electromagnetic radiation is irrelevant for the functioning of the method.

Alternatively or additionally, the at least one sensor has at least one wireless sensor, in particular at least one close-range sensor and/or at least one RFID sensor.

In a preferred embodiment, steps c), d) and e) of the method are performed by a device which comprises both the optical sensor, i.e. in particular a camera, and the electronic data processing device, in particular the microprocessor. This may be a tablet computer, a smartphone or another computer, for example.

It is advantageous if one, preferably several, but especially preferably all markings are arranged on the component in the form of stickers or magnetic elements, or are printed onto the components. Specifically, the use of magnetic elements with markings arranged thereon has the advantage that they can be removed from the components without a trace and used for another prosthesis during the next prosthesis set-up. However, the disadvantage is that the magnetic elements can only be arranged on materials that can be magnetized.

If the marking in step a) of the method is arranged on an existing component, for example by the orthopedic technician, it is difficult to maintain the exact position of the marking on the component so that the actual position and/or orientation of the component can be deduced from the detected position and/or orientation of the marking in step d) of the method. This can be resolved, for instance, by precisely specifying a field or position on the respective component within which the marking must be arranged. Alternatively or additionally, an electronic data processing device may be provided which renders it possible to first detect the component with the marking arranged thereon, for example optically, and to determine the position and/or orientation of the marking on the component. This results in a higher degree of data processing, but in principle increases the accuracy of the positioning of the marking on the respective component.

If the components feature a printed marking, which could be applied, for example, directly by the manufacturer of the components, these difficulties do not arise. However, the disadvantage in this case is that the markings cannot be removed from the component, or only with considerable effort, and are consequently also present on the finished prosthesis and may have visually disruptive effects.

In a preferred embodiment, the method is combined with the method from the prior art, in which the ground reaction forces and/or the center of gravity of the body of the person wearing the prosthesis are detected. The method can be carried out multiple times if, for example, the comparison between the actual position and/or actual orientation and the target position and/or target orientation shows large deviations, which are corrected. Steps c) to e) of the process can be performed again on the corrected prosthesis set-up. If after one or several of these interactions the prosthesis set-up reaches the desired quality, the patient can put on the prosthesis and undergo one of the methods already described. In this case too, steps c), d) and e) of the method can be performed alongside the methods of the prior art, wherein, for example, a camera of a device from the prior art is used as an optical sensor, as marketed by the applicant under the name “LASAR-Posture”, for example.

It is advantageous if at least one, but preferably multiple, especially preferably all markings are located on separate marking components, such as a rod or plate arranged on the respective component. This can be achieved, for instance, with velcro elements, tapes, magnetic elements and/or clamping elements. This embodiment has the advantage that the marking components on which the markings are situated can be easily removed from the respective component of the prosthesis such that no visually disruptive effects are left on the finished prosthesis. Preferably, the marking components are adapted in their geometric form to the geometric form of the respective component, so that they can preferably only be arranged on the respective component in a few, especially preferably only in one, position and orientation. On the one hand, this ensures that the marking components and thus the marking can be easily detached from the respective component and, on the other hand, that a fixed assignment between the position and/or the orientation of the marking and the position and/or the orientation of the component is rendered possible in a particularly simple manner.

It is advantageous if the target position and/or target orientation are read from a database. It is especially preferable if the target position and/or the target orientation is determined on the basis of the physical data and/or measurements of the wearer of the prosthesis, in particular the flexion contracture and/or heel height of a shoe. These data, measurements and length specifications as well as angles, if applicable, are recorded by the wearer of the prosthesis before the prosthesis is set up and fed into the database. In particular, the use of the flexion contracture allows the required tilting of the socket into the target position and/or the target orientation of the individual components to be taken into account and the comfort experienced when wearing the prosthesis to be increased.

It is advantageous if, based on the comparison between the actual position and/or the actual orientation of the components relative to each other with the target position and/or the target orientation, a recommendation for action is given via an output device if the actual position and/or the actual orientation deviates from the target position and/or the target orientation by more than a predetermined limit. This recommendation for action may, for example, consist of suggesting a displacement or an angular adjustment of one or more of the components. It is thus possible, especially for the orthopaedic technician, to quickly and safely use the adjustment option to achieve the desired result in the often complex set-up of a prosthesis made up of different components with a multitude of different and mutually influencing adjustment options. This is particularly advantageous if the prosthesis is set up together with the patient, as the time required for the prosthesis set-up procedure is significantly reduced.

Preferably, the marking also contains information about the component on which the marking has been provided and, in particular, is encoded therein. This renders it possible for the electronic data processing device to take into account information about the component used, for example for the recommended action, based on the sensor data. Instead of abstract recommendations for action, for example to move the knee joint relative to the socket by a certain distance in a certain direction, a specific recommendation for action could be given, for example asking the orthopaedic technician to turn a certain screw by a certain number of rotations in a certain direction. If information about the various adjustment options of the different components is also stored in the database and accessible to the electronic data processing device, the electronic data processing device preferably accesses this information if it also obtains information about the component used from the recorded sensor data. In addition, it is possible in this way to identify component combinations that should not be used together due to the technical characteristics of the different components, since, for example, the use of a particular prosthetic foot can cancel out or not really exploit the technical advantages of a prosthetic knee joint that is also to be used.

The marking is preferably a QR code, a barcode and/or an RFID.

In an especially preferred embodiment of the method, the position and/or orientation of the marking of the closely arranged components is detected from the different directions, particularly the sagittal and frontal direction. The comparison of the actual position and/or actual orientation of the components with the target position and/or target orientation is thus possible not only in one plane, but in two planes that are preferably perpendicular to each other, so that the prosthesis set-up can be completely monitored in three dimensions and corrected if necessary.

In order to draw conclusions from the detected position and/or orientation of the markings regarding the actual position and/or the actual orientation of the respective component, it is advantageous to also include the position of the optical sensor, in particular the camera, in the calculations. Inclined positions or displacements of the camera from the perpendicular relative to the sensor, in particular inclined camera positions, can be determined from the distortion of the detected markings and eliminated so that the applicable position and/or orientation of the markings and thus the applicable actual position and/or actual orientation of the components relative to each other can be determined.

The greater the extension of the respective marking in one direction, the easier the orientation of a marking is to determine. Preferably, the extension of the marking in a first spatial direction is greater, for example twice, three times or four times as great as in another spatial direction, which is, for example, perpendicular to the first spatial direction. For example, a QR code can be stretched in a first spatial direction without affecting the expansion in the other spatial direction.

Preferably, steps b) to e) are conducted several times in the method, wherein the arrangement of different components close to one another is also understood to mean the correction of this arrangement. Based on the comparison in step e) of the method, the recommendation for action is given via an output device, for example a monitor or display. The orthopedic technician preferably follows this recommendation for action and re-arranges the various components in relation to each other. The position and/or orientation of the markings can then be detected again and the actual position and/or actual orientation of the components determined from these; these can be compared with the target position and/or target orientation. If further deviations are determined, this leads to a renewed recommendation for action, which results in a renewed rearrangement or displacement of the different components in relation to each other. If a sufficient result has been achieved, the respective actual values are preferably saved. In this way, proof of a professionally performed prosthesis set-up can be provided, which is of particular interest to health insurance companies that are to provide full or at least partial reimbursement of costs.

If the prosthesis being set up includes, for example, a prosthesis socket, as is the case with upper leg prostheses, lower leg prostheses or forearm prostheses, this socket can preferably also be detected by the camera, i.e. the optical sensor. The correct 50/50 plane for the prosthesis set-up can be determined proximally and distally by the electronic data processing device using the sensor data of the optical sensor, wherein it is particularly preferable for the detected location to be indicated to the orthopedic technician, for example via a laser marking. Alternatively or additionally, the orthopedic technician can also apply at least one marking to the socket so that this can be detected by the optical sensor and processed by the electronic data processing device. The methods described here are especially well-suited for a static prosthesis set-up. Once this has been completed, however, it is advantageous to leave the markings on the respective components of the prosthesis and also use them for a gait analysis, i.e. in particular a dynamic prosthesis set-up. The patient is asked to perform a certain sequence of steps or different gait modes. This is observed with an optical sensor, in particular a camera, wherein the course of the position and/or the orientation of the markings in terms of time is tracked. In this way, the sensor data of this time-related course can be used for a dynamic gait analysis and thus for the dynamic prosthesis set-up. The actual values determined and preferably stored in a previously performed static prosthetic set-up can also be used for comparison. They can also be used to restore the prosthesis to its original state.

The invention also solves the problem by way of a device for conducting one of the methods described here, the device having at least one optical sensor for detecting a position and/or an orientation of the markings of the closely arranged components and an electronic or electric control system, in particular a data processing device, which is configured to carry out steps d) and e) of the method. It is advantageous if the device features an output device, for example a display, in which preferably both the image of the optical sensor, for example a camera image, is displayed as well as electronically calculated components, for example lines of force, detected joint axes and similar elements, from which the correct detection of the components can preferably be derived on the one hand and the recommendations for action understood on the other.

In the following, an example of an embodiment of the present invention will be explained in more detail by way of the attached figures:

They show:

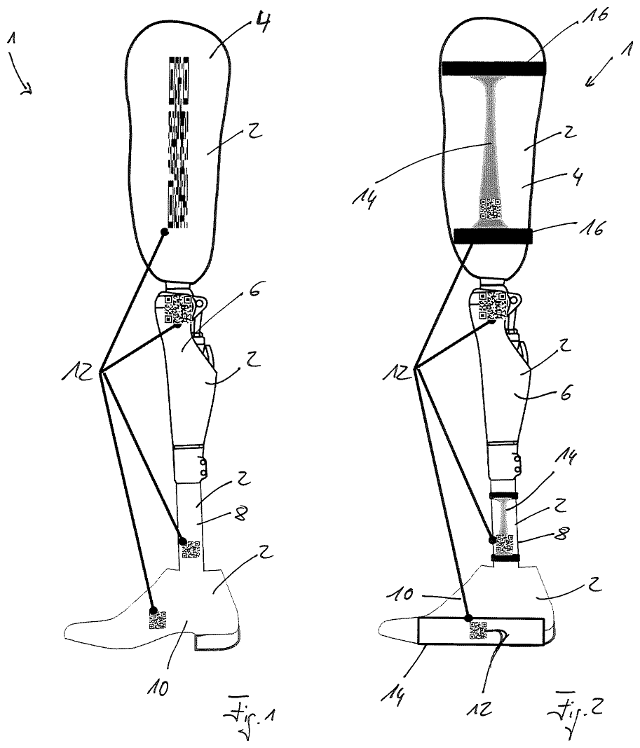

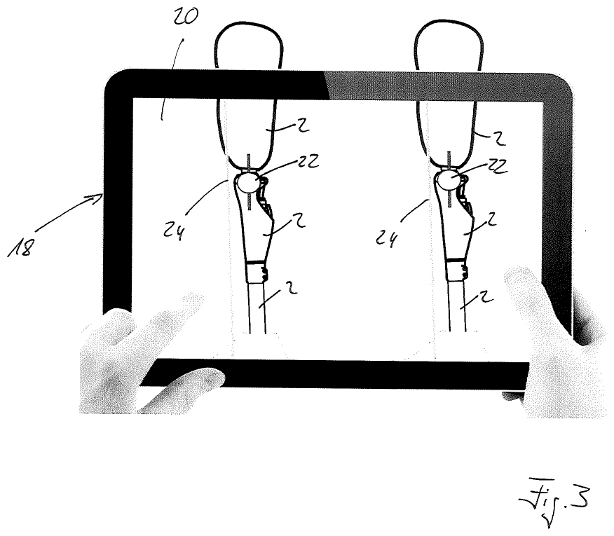

FIGS. 1 and 2—schematic representations of a leg prosthesis with markings for a method according to an example of an embodiment of the present invention,

FIG. 3—the schematic representation of a corresponding device.

FIG. 1 depicts a prosthesis 1 with various components 2. These are, from top to bottom, a prosthesis socket 4, a prosthetic knee joint 6, a lower leg element 8 and a prosthetic foot 10. Each of these components 2 features a marking 12 in the form of a QR code. Particularly in the case of the marking 12 arranged on the prosthesis socket 4, it is clear that the marking 12 can also be designed to be elongated. This facilitates the detection of an alignment and thus the orientation of both the marking 12 and the corresponding component 2.

FIG. 2 shows the prosthesis 1 with the previously specified components 2 and the markings 12; however, these are now designed differently. The marking 12 arranged on the prosthetic knee joint 6 is designed as shown in FIG. 1 and is, for example, printed on the prosthetic knee joint 6 or arranged on the element by means of a sticker. The marking 12 on the prosthetic foot 10 is arranged on a marking component 14, which is arranged on the prosthetic foot 10, for example via a magnetic interaction. Alternatively, the marking 12 can also be pushed onto the foot, wherein the position of the marking 12 can then be determined, for example, by end stops which are located, for example, on the forefoot and the heel. The markings 12 on the lower leg element 8 and on the prosthesis socket 4 are also arranged on the respective component via marking components 14. It can be seen particularly clearly on the prosthesis socket 4 that the marking component 14 is an elongated, plate-shaped element, which could be described as a rod, for example, on which the actual marking 12 is arranged. The elongated design of the marking 12 makes it easier to determine the orientation of the marking 12. The marking component 14 is arranged on the respective component 2 via two fastening strips 16, which may, for example, be in the form of straps, velcro fastening elements or adhesive elements. Again, the elongated shape of the marking component 14 facilitates the detection of the orientation of the marking component 14 and thus of the marking 12.

FIG. 3 schematically depicts a device 18 for performing one of the methods described here, said device being designed as a tablet. An output device 20 in the form of a display is shown on the one hand as a camera image of the detected prosthesis with the components 2 and on the other hand by the symbols 22 where markings have been detected. The symbol 22 includes a circle representing the position of the detected marking and two dash elements extending upwards and downwards in the example of an embodiment shown representing the orientation of the detected marking.

In addition, a force curve 24 is shown, which has been calculated either from the determined actual positions and/or actual orientations of the components 2 relative to each other or determined, for example, on the basis of force sensors or pressure sensors in a base plate, not shown, from the prior art. By means of the device 18 in the embodiment shown, it is no longer necessary to project detected or calculated lines onto the prosthesis itself or even onto the patient. It is sufficient for the calculated or detected lines to be displayed in the output device 20 of the device 18.

### REFERENCE LIST

1 prosthesis
2 component
4 prosthesis socket
6 prosthetic knee joint
8 lower leg element
10 prosthetic foot
12 marking
14 marking component
16 fastening strips
18 device
20 output device
22 symbol
24 force curve

1. A method for carrying out a prosthesis set-up, wherein multiple components are arranged close to each other, the method comprising the steps of:
a) providing at least one marking on at least one component;
b) arranging various components close to each other;
c) detecting a position and/or an orientation of the at least one marking of the closely arranged components by means of at least one sensor;
d) determining the actual position and/or the actual orientation of the closely arranged components relative to one another using the detected position and/or orientation of the at least one marking; and
e) comparing the determined actual position and/or actual orientation with a target position and/or target orientation.

2. The method according to claim 1, wherein the prosthesis set-up is a static prosthesis set-up.

3. The method according to claim 1, wherein the prosthesis is set up according to a set-up recommendation that designates at least one component as relevant for adjustment, wherein preferably at least one marking is provided on each of the components designated as relevant for adjustment.

4. The method according to claim 1, wherein the at least one sensor comprises at least one optical sensor, preferably a camera, especially preferably for visible light or infrared radiation.

5. The method according to claim 1, wherein at least one, preferably several, but preferably all, markings are arranged on the components in the form of stickers or magnetic elements, or are printed onto the components.

6. The method according to claim 1, wherein at least one, but preferably several, especially preferably all markings are located on separate marking components, such as a rod or plate arranged on the respective component.

7. The method according to claim 6, wherein the marking components are detachably arranged on the respective component, in particular by means of velcro elements, tapes, magnets and/or clamping elements.

8. The method according to claim 1, wherein the target position and/or target orientation are read from a database.

9. The method according to claim 1, wherein the target position and/or the target orientation is determined on the basis of the physical data and/or measurements of the wearer of the prosthesis, in particular the flexion contracture and/or heel height of a shoe.

10. The method according to claim 1, wherein, based on the comparison, at least one recommendation for action is given via an output device if the actual position and/or the actual orientation deviates from the target position and/or the target orientation by more than a predetermined limit.

11. The method according to claim 1, wherein the marking also contains, and in particular encodes, information about the component on which the marking has been provided.

12. The method according to claim 1, wherein that the markings are a QR code, a barcode and/or an RFID.

13. The method according to claim 1, wherein the position and/or orientation of the markings of the closely arranged components are detected from two different directions, particularly the sagittal and frontal directions.

14. A device for performing a method according to claim 1, wherein the device has at least one sensor for detecting a position and/or an orientation of the at least one marking of the closely arranged components and an electrical and/or electronic control system, in particular an electronic data processing device, which is configured to carry out steps d) and e) of the method.

15. A method for carrying out a prosthesis set-up comprising the steps of:
a) providing at least two prosthesis components;
b) providing at least one marking on at least one prosthesis component;
c) arranging the prosthesis components close to each other;
d) detecting a position and/or an orientation of the at least one marking of the closely arranged at least one prosthesis component by means of at least one optical sensor;
e) determining the actual position and/or the actual orientation of the closely arranged prosthesis components relative to one another using the detected position and/or orientation of the at least one marking; and
f) comparing the determined actual position and/or actual orientation with a target position and/or target orientation, the target position and/or target orientation being obtained from a database.

16. The method of claim 15, wherein the at least one marking is arranged on the at least one prosthesis component in the form of a sticker, a magnetic element, or a marking printed onto the at least one component.

17. The method according to claim 15, wherein the at least one marking is located on a separate marking component such as a rod or plate arranged on the at least one prosthesis component.

18. The method according to claim 15, wherein the marking encodes information about the component on which the marking has been provided.

19. The method according to claim 15, wherein the position and/or orientation of the markings of the closely arranged components are detected from at least two different directions, particularly the sagittal and frontal directions.

20. A method for carrying out a prosthesis set-up comprising the steps of:
g) providing at least two prosthesis components;
h) providing at least one QR code, barcode and/or RFID on at least one of the prosthesis components;
i) arranging the prosthesis components close to each other;
j) detecting a position and/or an orientation of the at least one marking of the closely arranged at least one prosthesis component by means of at least one optical sensor;
k) determining the actual position and/or the actual orientation of the closely arranged prosthesis components relative to one another using the detected position and/or orientation of the at least one marking;
l) comparing the determined actual position and/or actual orientation with a target position and/or target orientation, the target position and/or target orientation being obtained from a database; and
m) providing at least one recommendation for action if the actual position and/or actual orientation deviates from the target position and/or the target orientation by more than a predetermined limit.
